(19) United States
(12) Patent Application Publication
(10) Pub. No.: US 2022/0221453 A1
(43) Pub. Date: Jul. 14, 2022

(54) TEST PIECE FOR RAPID AND QUANTITATIVE DETECTION OF TISSUE CELL PROTEINS

(71) Applicants: Jun MEI, Shanghai (CN); Jieying ZHANG, Shanghai (CN); Mingjun WANG, Shanghai (CN)

(72) Inventors: Jun MEI, Shanghai (CN); Jieying ZHANG, Shanghai (CN); Mingjun WANG, Shanghai (CN)

(21) Appl. No.: 17/612,126

(22) PCT Filed: May 15, 2020
(86) PCT No.: PCT/CN2020/090701

§ 371 Date: Nov. 17, 2021

(30) Foreign Application Priority Data
May 17, 2019 (CN) 201910414499.3

(51) Int. Cl. G01N 33/543; G01N 33/574; B01L 3/00

(52) U.S. Cl. CPC G01N 33/54388; B01L 2300/0825; B01L 3/5023; G01N 33/57415

(57) ABSTRACT

A test strip for the rapid quantitative detection of tissue cellular proteins, comprising a substrate, and a sampling area, a colloidal gold area, a protein detection area, and a water absorption area are positioned on the substrate in sequence from one end of the substrate to the other end; the protein detection area comprises a plurality of detection points, the detection points being arranged in multiple columns, the circle centers of the detection points in each column being connected to one other to form a straight line, and the straight line being perpendicular to the flow direction of the sample to be tested; the detection points on the protein detection area present an alternating array. The present test strip can rapidly and accurately detect tissue cellular proteins qualitatively and quantitatively.

### TECHNICAL FIELD

The present invention relates to detection field, and in particular to test strip for rapid and quantitative detection of tissue cell proteins.

### TECHNICAL BACKGROUND

Due to the extensive drug effects, the previous tumor treatments, especially chemotherapy, kill a large number of normal cells while killing tumor cells, thereby resulting in intolerable, toxic side effects. With the rapid development of medical research, scientists have gradually developed targeted tumor therapy drugs with good specificity and little toxic and side effects. At present, this kind of targeted therapy has become the trend of tumor treatment because of its good therapeutic effect and less toxic side effects.

Surgery or biopsy may accelerate the infiltration and spread of tumor cells, and it is recognized in medical field that timely and effective treatment is a necessity to prevent the spreading. However, it remains a trouble unsolved as how to determine the molecular type of tumor quickly and accurately in clinic, so as to further determine whether the patient is suitable for a certain targeted therapy.

Therefore, it is urgent in the art to develop a rapid and quantitative method to test the cellular protein level in tissues, especially the cellular protein level related to tumor cells, so as to provide guidance for treatment of diseases.

### SUMMARY OF INVENTION

An object of the present invention is to provide a test strip for rapid, quantitative testing of tissue cellular proteins, especially cellular proteins associated with tumor cells.

In the first aspect of the present invention, it provides a test strip which comprises a substrate, wherein a sampling area, a colloidal gold area, a protein testing area and a water absorption area are arranged on the substrate in sequence from one end to the other end of the substrate;

the protein testing area comprises a plurality of testing points which are arranged in a plurality of lines, wherein the centers of the testing points in each line are connected with each other to form a straight line which is perpendicular to a flow direction of a sample to be detected; and

the testing points in the protein testing area are arranged as an alternating array.

In another preferred embodiment, the testing points are arranged in n+1 lines, where n is 3-30, preferably 3-20, preferably 3-15, preferably 5-12, and n is a positive integer (e.g. 1, 2, 3, 4, 9, 15 or 30, etc.).

In another preferred embodiment, the shape of each testing point is same or different.

In another preferred embodiment, the shape of the testing point is circular.

In another preferred embodiment, the inner diameter of the testing points are same or different.

In another preferred embodiment, the center distance of two adjacent testing points in each line is same.

In another preferred embodiment, the number of testing points contained in each line is 2-30, preferably 2-20, more preferably 2-10, and most preferably 2-6.

In another preferred embodiment, the number of testing points in each line differs by 1-2.

In another preferred embodiment, the centers of each line of testing points are connected to each other to form straight lines which are parallel to each other.

In another preferred embodiment, the vertical distance between two adjacent lines is 1.0-1.8 mm, preferably 1.2-1.6 mm, and more preferably 1.3-1.5 mm.

In another preferred embodiment, the distance between the center of two adjacent testing points in one line and one testing point in another adjacent line corresponding to the gap between them is equal or unequal.

In another preferred embodiment, the distance between the center of two adjacent testing points in one line and one testing point in another adjacent line corresponding to the gap between said two adjacent testing points in the line is 1.3-2.1 mm, preferably 1.4-1.8 mm, and more preferably 1.6-1.8 mm, respectively.

In another preferred embodiment, the distance between the center of two adjacent testing points in one line and one testing point in another adjacent line corresponding to the gap between the adjacent two testing points is equal or unequal.

In another preferred embodiment, a gap between two adjacent testing points D1, D2 corresponds to a testing point D3 of another adjacent line, and the distance between the centers of the two testing points D1 and D3 is 1.3-2.1 mm, preferably 1.4-1.8 mm, and more preferably 1.6-1.8 mm. The distance between the centers of the two testing points D2 and D3 is 1.3-2.1 mm, preferably 1.4-1.8 mm, and more preferably 1.6-1.8 mm.

In another preferred embodiment, an equilateral triangle is formed between two adjacent testing points in one line and one testing point in another adjacent line corresponding to a gap between said two adjacent testing points in the line.

In another preferred embodiment, each inner diameter of the testing points is 0.5-1.2 mm, preferably 0.6-1.0 mm, and more preferably 0.7-0.9 mm.

In another preferred embodiment, the distance between the centers of two adjacent testing points in each line is 1.3-2.1 mm, preferably 1.4-1.8 mm, and more preferably 1.6-1.8 mm.

In another preferred embodiment, the distance between the center of a circle between two adjacent testing points in one line and one testing point in another adjacent line corresponding to the gap therebetween is 1.3-2.1 mm, preferably 1.4-1.8 mm, and more preferably 1.6-1.8 mm, respectively.

In another preferred embodiment, the test strip further comprises a quality control area located on the substrate.

In another preferred embodiment, the quality control area is located between the protein testing area and the water absorption area.

In another preferred embodiment, the colloidal gold area is loaded with one or more complexes selected from the group consisting of: colloidal gold antibody complex, colloidal silver antibody complex, and a combination thereof

The colloidal gold antibody complex is formed by coupling colloidal gold with an antibody selected from the group consisting of: a monoclonal antibody targeting a tissue cellular protein to be detected, polyclonal antibody targeting the tissue cellular protein to be detected, and a combination thereof.

The colloidal silver antibody complex is formed by coupling colloidal silver with an antibody selected from the group consisting of: a monoclonal antibody targeting a tissue cellular protein to be detected, polyclonal antibody targeting tissue cellular protein to be detected, and a combination thereof.

In another preferred embodiment, the colloidal gold area is provided with glass fibers on which the complex is coated.

In another preferred embodiment, the tissue cellular protein to be detected is HER2 protein.

In another preferred embodiment, the colloidal gold area is further loaded with an IgG colloidal gold complex, an IgG colloidal silver complex, or a combination thereof.

In another preferred embodiment, the testing point of the protein testing area is loaded with one or more antibodies selected from the group consisting of: a monoclonal antibody against a tissue protein to be detected and polyclonal antibodies against a tissue protein to be detected.

In another preferred embodiment, the colloidal gold area comprises a colloidal gold complex loaded with a HER2 antibody, and the testing area comprises an anti-HER2 antibody.

In another preferred embodiment, the protein testing area is provided with a nitrocellulose membrane, on which the monoclonal antibody against the tissue protein to be detected or the polyclonal antibody against the tissue protein to be detected is coated.

In another preferred embodiment, the HER2 antibody in the HER2 antibody colloidal gold complex is a rabbit-derived, anti-human HER2 antibody.

In another preferred embodiment, the sampling area comprises a sampling pad.

In another preferred embodiment, the water absorption area comprises water absorption paper.

In the second aspect of the invention, it provides a test kit for testing tissue cellular protein, which comprises:

(i) the test strip for testing tissue cellular protein according to the first aspect of the present invention;

(ii) a specification.

In another preferred example, in the specification, it describes the tissue cellular protein test by using the test strip for tissue cellular protein test according to the first aspect of the present invention.

In the third aspect of that invention, it provides the use of the test strip according to the first aspect of the present invention for detecting tissue cellular protein.

In another preferred embodiment, the detection is in vitro.

In another preferred embodiment, the detection comprises qualitative and/or quantitative detection.

In another preferred embodiment, the detection is an auxiliary detection.

In another preferred embodiment, the detection is non-therapeutic and non-diagnostic.

In the fourth aspect, it provides a method for detecting tissue cellular protein, which comprises: detecting a tissue cellular protein in a sample using the test strip of the first aspect of the present invention for testing tissue cellular protein

In another preferred embodiment, the detection is in vitro.

In another preferred embodiment, the detection comprises qualitative and/or quantitative detection.

In another preferred embodiment, the detection is an auxiliary detection.

In another preferred embodiment, the detect is non-therapeutic and non-diagnostic.

In another preferred embodiment, the sample includes cell lysate, tissue lysate, plasma, serum, whole blood, urine or sputum.

It should be understood that within the scope of the present invention, the above-described technical features of the present invention and the technical features described in detail below (e.g., embodiments) may be combined with each other to constitute a new or preferred technical solution which is not redundantly repeated one by one due to the limitation of space.

### DRAWINGS

FIG. 1 is a schematic diagram of the structure of a test strip according to embodiment 1.

FIG. 2 shows positive result of HER2 protein standard solutions with different concentrations, displaying as chromogenic protein testing points of the test strip. In the order of serial numbers 1-5, the concentrations of HER2 protein standard solutions are 4 ng/ml, 20 ng/ml, 100 ng/ml, 500 ng/ml and 2500 ng/ml, respectively.

FIG. 3 shows the standard curve for HER2 protein standard solutions with different concentrations with their corresponding total absorbance values. The standard curves are shown in FIG. 2. The correlation coefficient of the standard curve R² is 0.99.

FIG. 4 shows the testing results of immunohistochemistry.

FIG. 5 shows the testing results of protein imprinting.

FIG. 6 shows testing results of HER2 proteins from different subjects by using the test strip of embodiment 1.

FIG. 7 shows the test results of test strip of embodiment 1 and that of Comparative Example 1, both of which are tested with HER2 protein standard solutions of the same concentration. The left panel shows the testing results of Comparative Example 1 wherein testing points are arranged in a parallel way. The right panel shows the testing results of embodiment 1 wherein the testing points are arranged in an alternating array.

### EMBODIMENTS

After extensive and intensive researches and numerous screenings and trials, the inventors have unexpectedly discovered a test strip capable of rapid, quantitative detection of tissue cellular protein. On the test strip of the present invention, the testing points in the protein testing area are in a specific arrangement. The test strip can be used for a rapid, quantitative detection of tissue cellular protein with high accuracy. On this basis, the inventors completed the invention.

### Terms

Unless otherwise defined, all technical and scientific terms used herein have the same meanings as would normally be understood by those of ordinary skill in the art to which the invention pertains.

As used herein, the terms “comprise”, “include” and “contain” can be used interchangeably to include not only closed definitions, but also semi-closed, and open definitions. In other words, these terms include “consist of” and “substantially consist of”.

As use herein, the term “HER2 protein” and “proto-oncogene human Epidermal growth factor receptor 2 (human epidermal growth factor receptor-2, HER2)” can be used interchangeably. HER2 is the target protein of breast cancer targeted therapy. For HER2 positive (overexpressed or amplified) breast cancer, Herceptin and other targeted drugs targeting HER2 have a good therapeutic effect, and the treatment mode thereof is quite different from that for other types of breast cancer.

Test Strip

The invention provides a test strip for testing tissue cellular protein, which can be used for rapid, qualitative and quantitative detection of the level of tissue cellular protein, especially the level of cellular proteins related to tumor cells.

The test strip of the present invention can also be referred to as a reagent strip or a test strip, etc.

Typically, the test strip comprises a substrate, and a sampling area, a colloidal gold area, a protein testing area and a water absorption area are positioned on the substrate in sequence from one end of the substrate to the other end.

The protein testing area comprises a plurality of testing points which are arranged in a plurality of lines. The centers of the testing points in each line can be connected with each other to form a straight line, which is perpendicular to the flow direction of the sample to be detected;

The testing points in the protein testing area are arranged as an alternating array.

In the present invention, it should be understood that the alternating array means that the testing points of two adjacent lines are alternately arranged. In other words, the alternating array means that the gap between two adjacent testing points in one line corresponds to another testing point in another adjacent line.

In the present invention, the substrate can also be referred to as a base, backing, etc., which plays a supporting function.

In the invention, the flow direction of the sample to be detected is from the sampling area to the water absorption area, and is perpendicular to the straight line formed by the connection between the centers of testing points on each line.

Sampling Area

In the test strip of the present invention, the sampling area is used for adding samples to be detected. The sampling area may include a sampling pad. Preferably, the sample pad is a fibrous membrane.

In another preferred embodiment, the sample to be detected may include cell lysate, tissue lysate, plasma, serum, whole blood, urine or sputum. Preferably, the sample to be detected contains tissue cellular proteins, for example HER2 protein.

Colloidal Area

In the test strip of the present invention, the colloidal gold area may contain a gold reaction pad.

The colloidal gold area may be loaded with one or more complexes selected from the group consisting of: colloidal gold antibody complex, colloidal silver antibody complex or a combination thereof.

In another preferred embodiment, the colloidal gold antibody complex is formed by conjugating the colloidal gold with an antibody selected from the group consisting of: a monoclonal antibody against tissue cellular protein to be detected, polyclonal antibody against tissue cellular protein to be detected, or a combinations thereof.

In another preferred embodiment, the colloidal silver antibody complex is formed by conjugating the colloidal silver with an antibody selected from the group consisting of: a monoclonal antibody against tissue cellular protein to be detected, polyclonal antibodies of tissue cellular protein to be detected, or a combination thereof.

In another preferred embodiment, the tissue cellular protein to be detected is HER2 protein.

In another preferred embodiment, the colloidal gold area is further loaded with an IgG colloidal gold complex.

In another preferred embodiment, the colloidal gold area is provided with glass fibers, and the complex is coated on the glass fibers.

Protein Testing Area

In the test strip of the present invention, the specific structure of the protein testing area enables test strip to detect the protein level of tissue cellular protein rapidly, qualitatively and quantitatively,

In the present invention, the shape of the testing point is not particularly limited as long as effect of the present invention is achieved, and a preferred shape of the testing point comprises (but is not limited to) a circle, a square, or a combination thereof. More preferably, the shape of the testing point is circular.

In this application, it should be understood that if the shape of the testing point is not circular, it is necessary to simulate the testing point as a circle when defining the characteristics such as the inner diameter of the testing point. The specific simulation process is as follows: two points with longest distance in the shape of the testing point are taken, and the distance is taken as diameter to simulate as circle.

In the present invention, the inner diameter of the testing point refers to the diameter of the testing point, and when the testing point is circular, the inner diameter refers to the diameter of the circular testing point.

In a preferred embodiment of the invention, the inner diameter of the testing point is 0.5-1.2 mm, preferably 0.6-1.0 mm, and more preferably 0.7-0.9 mm.

In another preferred embodiment, the distance between the centers of two adjacent testing points in each line is 1.3-2.1 mm, preferably 1.4-1.8 mm, and more preferably 1.6-1.8 mm.

In another preferred embodiment, the distance between the center of a circle between two adjacent testing points in one line and one testing point in another adjacent line corresponding to the gap therebetween is 1.3-2.1 mm, preferably 1.4-1.8 mm, and more preferably 1.6-1.8 mm, respectively.

In another preferred embodiment, the testing points are arranged in n+1 lines, wherein n=3-30, preferably 3-20, more preferably 3-15, still more preferably 5-12, and n is a positive integer (e.g. 1, 2, 3, 4, 9, 15 or 30, etc.).

In another preferred embodiment, the vertical distance between two adjacent lines is 1.0-1.8 mm, preferably 1.2-1.6 mm, and more preferably 1.3-1.5 mm. In the present invention, the vertical distance between two adjacent lines refers to the vertical distance of straight lines formed by connecting the centers of testing points of two adjacent lines. In other words, “vertical distance between two adjacent lines” refers to the vertical distance between a straight line (L1) formed by connecting the centers of one line of testing points and a straight line (L2) formed by connecting the centers of another adjacent line of testing points, that is, the vertical distance between a straight line (L1) and a straight line (L2).

Quality Control Area

The test strip of the invention may also include a quality control area located on the substrate. The quality control area may be provided with a quality control line, preferably perpendicular to the direction of flow direction. Preferably, the quality control area is located between the protein testing area and the water absorption area.

Quality control lines can be used to indicate whether the testing process (for example, chromatography process) is normal. For example, in the test strip of the present invention, the sampling area is coated with the colloidal gold complex of IgG, and the quality control line is coated with the antibody of IgG, and the IgG and the antibody of IgG play the role of quality control, indicating whether a certain testing process (chromatography process) is normal or not. If the IgG can normally reach the quality control line through chromatography, the antibody of the IgG on the quality control line is combined with the IgG and color will be displayed. However, if the testing piece is failed or damaged, the IgG cannot reach the quality control line normally, and the color cannot be displayed.

Therefore, those skilled in the art can choose the IgG for the present invention. In specific embodiments, the IgG includes, but is not limited to, rabbit IgG, horse IgG, mouse IgG, and the like. Accordingly, the skilled in the art can choose the IgG antibody for use in the present invention as long as the IgG antibody is an IgG antibody. In specific embodiments the IgG antibodies include but are not limited to IgG of goat against rabbit, IgG of goat against horse, and the like.

In the present invention, the sampling area, the colloidal gold area, the protein testing area and the water absorption area are located on a substrate.

Water Absorption Area

In the present invention, the water absorption area may include water absorption paper. The water absorption area drives the sample to be detected from the sampling area to flow through the colloidal gold area and the protein testing area in turn to reach the water absorption area, thus ensuring the detection goes on smoothly.

The Main Advantages of the Present Invention Include:

1. The test strip of the present invention may be used to carry out a qualitative and quantitative test of tissue cellular protein rapidly and accurately (for example, cancer tissue cellular protein HER2) and the like, and has high sensitivity, thus greatly improving disease diagnosis and treatment effects.

2. In the prior art, the testing of tissue cellular protein (such as cancer tissue cellular protein HER2) is mainly carried out by high performance liquid chromatography-tandem mass spectrometry, high performance liquid chromatography, biochip technology and the like, but these technologies have obvious defects, such as complex operation, expensive equipment as well as time-consuming, unsuitable for in situ testing and so on. In addition, the method for testing tissue cellular protein (such as cancer tissue cellular protein HER2) in the prior art requires more on instruments and operators and occupies a long time, thereby causing higher cost. The test strip of the present invention has the advantages of simple operation, low cost and high speed of testing, and overcomes deficiencies in the prior art such as the complicated operation, requirement for time-consuming and labor-consuming instrument and equipment.

The present invention is further described below accompanying with specific embodiments. It should be understood that these embodiments are intended to illustrate the invention only and are not intended to limit the scope of the invention. In the following embodiments, the test methods without specific conditions are usually in implemented with conventional conditions or conditions recommended by the manufacturer. Unless otherwise stated, percentages and proportions are calculated by weight.

### Embodiment 1

Test Strip

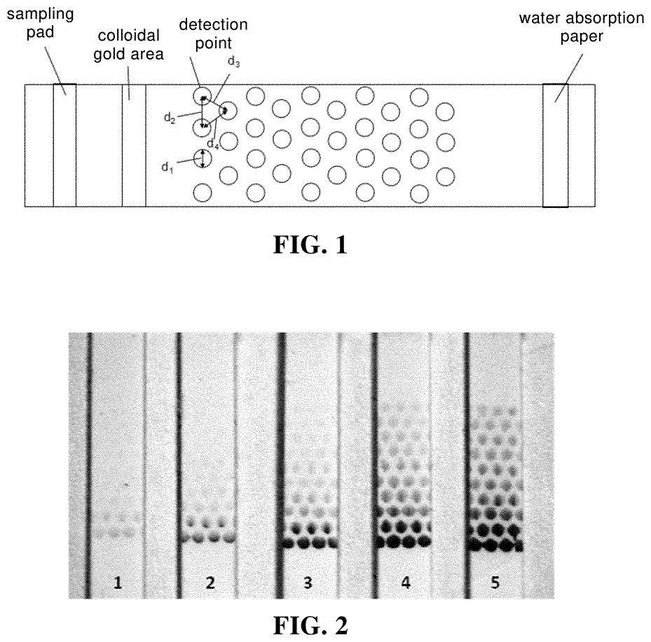

The structural schematic diagram of the test strip is shown in FIG. 1. The test strip comprises a substrate. From one end to the other end of the substrate, a sampling pad, a colloidal gold area, a protein testing area and a water absorption paper are arranged on the substrate in sequence. After the sample to be detected is added to the sampling pad, it flows through the colloidal gold area, the protein testing area and the water absorption paper in sequence;

Colloidal gold area: Colloidal gold area is loaded with colloidal gold complex of HER2 antibody, wherein the complex is coated on glass fiber;

Protein testing area: The protein testing area contains a plurality of testing points, and the testing points are loaded with mouse antibodies against HER2, which are fixed on nitrocellulose membranes. All testing points have the same circular shape. The testing points are arranged in a plurality of lines, wherein centers of testing points on each line are connected with each other to form a straight line. The straight line is perpendicular to the flow direction of the sample to be detected. The testing points on the protein testing area are presented as an alternating array, and the center distance of the two adjacent testing points in each line is identical. The straight line formed by connecting the centers of each line of testing points is parallel to each other. The inner diameter d1 of each testing point is 0.8 mm. The distance d2 between the centers of adjacent testing points in each line is 1.66 mm, and the distance between the centers of two adjacent testing points in one lines and one testing point in another adjacent liens corresponding to the gap between them is D3 or D4, wherein D3=1.66 mm and D4=1.66 mm, respectively.

### Embodiment 2

Quantitative Testing of HER2 Protein

HER2 protein standard solutions with different concentrations are tested using test strip of embodiment 1. Specific testing steps are as follows. The HER2 protein standard solution was diluted into 4 ng/ml, 20 ng/ml, 100 ng/ml, 500 ng/ml and 2500 ng/ml with phosphate buffer in proportion. Then 100 μl diluents of HER2 protein standard solution with different concentrations were added to the sampling area of the test strip. 5 minutes later, the positive result displayed as the chromogenic protein testing points of the test paper of HER2 protein standard solution of different concentrations, as shown in FIG. 2. In FIG. 2, the concentration of HER2 standard protein is 4 ng/ml, 20 ng/ml, 100 ng/ml, 500 ng/ml, 2500 ng/ml corresponding to sequence number 1-5. Using absorbance scanning detector to count the number of chromogenic testing points showing positive result and their absorbance values of each test strip, and to calculate the total absorbance value (Ab525 nm), which is the sum of the absorbance values of all testing points for 525 nm wavelength light, as shown in Table 1. Then the standard curve for HER2 protein standard solution of different concentrations corresponding to their total absorbance value (Ab525 nm) is drawn. The standard curve is shown in FIG. 3.

TABLE 1
Corresponding relationship between HER2
concentration and total absorbance value

| | HER2 Concentration (C) | | | | |
|---|---|---|---|---|---|
| | 4 | 20 | 100 | 500 | 2500 |
| | ng/ml | ng/ml | ng/ml | ng/ml | ng/ml |
| Total absorbance | 5 | 17 | 67 | 438 | 1709 |
| value (Ab) | | | | | |

A = 0.68 C + 22.10, correlation coefficient R² = 0.9966

As seen from Table 1 and the standard curves in FIG. 3, the test strip can accurately and quantitatively detect the HER2 Protein standard solution. Therefore, the test strip of embodiment 1 can be used for quantitative detecting of HER2 Protein concentration, and quantitative results with high accuracy can be achieved.

### Embodiment 3

In embodiment 3 the expression of HER2 protein of 5 Breast Carcinoma subjects are tested via immunohistochemistry, Western blotting and test strip of embodiment 1. Comparing with the accuracy of test of HER2 protein expression in Breast Carcinoma by the test strip of embodiment 1, wherein immunohistochemical method was a customary means in clinical testing, and Western blotting is a third-party standard. In immunohistochemistry, Western blotting and the testing method of embodiment 1, HER2 Proteins from tissue of 5 Breast Carcinoma subjects (respectively labeled as 1, 2, 3, 4 and 5) were tested, wherein samples with one serial number refer to breast cancer tissue from same subject.

Immunohistochemistry Test

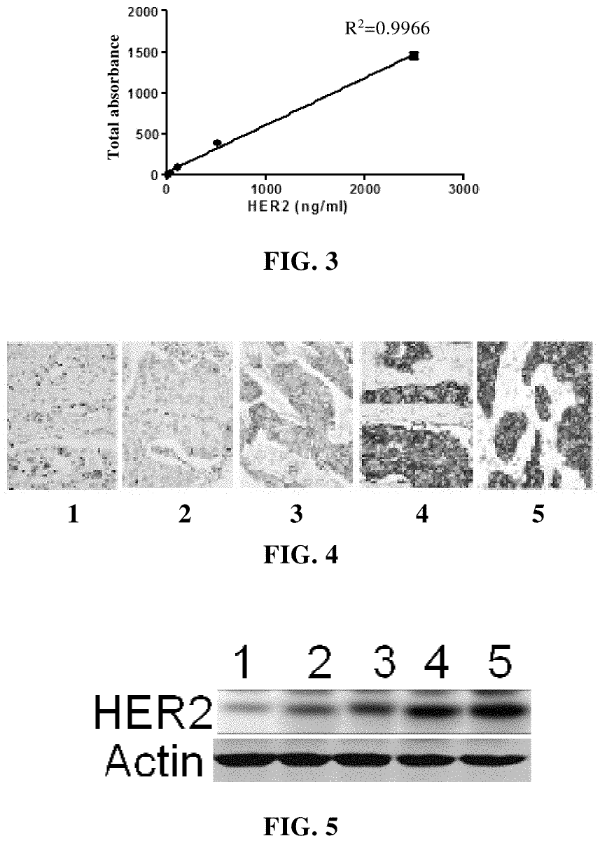

The tumor specimen after operation was fixed with formaldehyde and embedded in paraffin. After slices were dewaxed, hydrated, permeated and sealed, HER2 antibody from mice was added, and finally incubated with horseradish-peroxidase-labeled, IgG secondary antibody of sheep against mouse. Finally, color was displayed and a better photography under microscope was taken. The darker the color, the higher the expression of the tested protein. Samples of the same tissue from different subjects were tested by immunohistochemistry. The testing results are shown in FIG. 4.

Gold Standard Western Blotting Test

The total protein were obtained from tumor samples by tissue homogenization, cell breaking via SDS and extraction. After measuring the protein concentration, the same mass of total protein was taken, denatured by heating, separated by 8% SDS-polyacrylamide gel, and transferred onto 0.45 μm nitrocellulose membrane, which was blocked, and finally incubated with primary antibody and fluorescein-labeled secondary antibody. the images were scanned. Samples of the same tissue from different subjects were detected via Western blotting. The testing results are shown in FIG. 5.

Test Strip Test of Embodiment 1

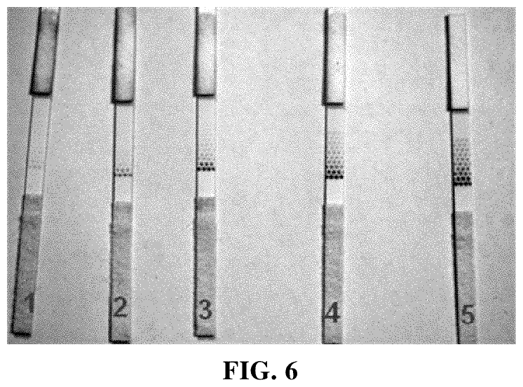

test strip of embodiment 1 was used to test HER2 protein expression level from breast cancer tissues of 5 subjects. Specific steps were as follows: approximately 0.1 g fresh breast cancer fresh tissues was taken and cut into pieces. Then 250 μl (microliter) protein lysis buffer was added for complete lysis in combination with grinding. After heating at 95 degree celcius for 5 minutes, and the lysate was immediately cooled on ice. Then 50 μl tissue lysate was taken and added to the sampling area of the test strip. 5 minutes later, the protein expression results of different subjects were as shown in the FIG. 6.

As can be seen in FIGS. 4-6, difference exists between the test results of the test strip of Example 1 and that of conventional immunohistochemistry, but it is consistent with the results of the third-party gold standard (Western blotting assay). The test results of sample 4 and sample 5 showed that the trend of immunohistochemistry was obviously inconsistent with the results of gold standard protein imprinting. However, the test results of the test strip of embodiment 1 is highly consistent with that of Western blotting. Thus, it can be seen that the test strip of embodiment 1 achieves a higher accuracy than that of the traditional immunohistochemistry. It can be seen that the values of different samples tested by the test strip of embodiment 1 are more consistent with the results of Western blotting assay, thus indicating that the test strip of embodiment 1 achieves a higher accuracy than that of the traditional immunohistochemistry.

### Comparison Example 1

Comparison Example 1 provides a test strip which differs from embodiment 1 in that: testing points on protein testing area were presented as parallel array.

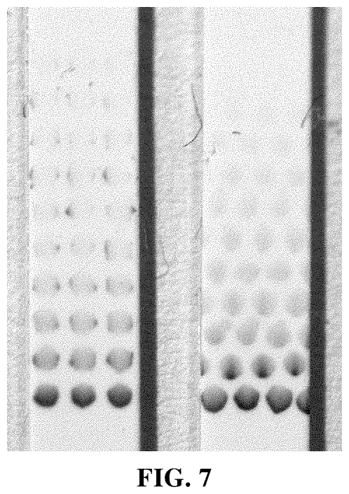

Using the method of quantitative test of HER2 protein of embodiment 2, the test result of the same concentration of HER2 protein standard solution by the Example 1 test strip and that of comparison example 1 were shown in FIG. 7.

As can be seen in FIG. 7, the number of positive chromogenic testing points of the test strip of comparative example 1 is significantly smaller than that of the test strip of embodiment 1. The more the chromogenic testing points, the better the accuracy and sensitivity of the measurement structure. The test strip of embodiment 1 is obviously superior to the test strip of Comparative Example 1. The total absorbance values obtained by the absorbance scanning detector were 2170 and 2930, respectively (the larger the absorbance value, the higher the accuracy and sensitivity), and the total absorbance value is the sum of the absorbance values of all the testing points at 525 nm wavelength light.

All documents referred to in the present invention are incorporated by reference herein as if each document were individually incorporated by reference. Further, it should be understood that upon reading the foregoing lecture of the present invention, various modifications or modifications may be made to the present invention by those skilled in the art, the equivalents of which also fall within the scope defined by the appended claims of the present application.

1. A test strip for testing tissue cellular protein, which comprises a substrate, and a sampling area, a colloidal gold area, a protein testing area and a water absorption area are arranged on the substrate in sequence from one end to the other end of the substrate;
the protein testing area comprises a plurality of testing points, and the testing points are arranged in a plurality of lines, wherein the centers of the testing points in each line connected with each other to form a straight line which is perpendicular to a flow direction of a sample to be detected; and
the testing points in the protein testing area are arranged as an alternating array.

2. The test strip according to claim 1, wherein the inner diameter of the testing point is 0.5-1.2 mm, preferably 0.6-1.0 mm, and more preferably 0.7-0.9 mm.

3. The test strip according to claim 1, wherein the distance between the centers of two adjacent test points in each line is 1.3-2.1 mm, preferably 1.4-1.8 mm, and more preferably 1.6-1.8 mm.

4. The test strip according to claim 1, wherein the distance between the center of two adjacent testing points in one line and one testing point in another adjacent line corresponding to the gap therebetween is 1.3-2.1 mm, preferably 1.4-1.8 mm, and more preferably 1.6-1.8 mm, respectively.

5. The test strip of claim 1, wherein the test strip further comprises a quality control area located on the substrate.

6. The test strip according to claim 1, wherein the colloidal gold area is loaded with one or more complexes selected from the group consisting of colloidal gold antibody complex, colloidal silver antibody complex and a combination thereof;
the colloidal gold antibody complex is formed by conjugating colloidal gold with an antibody selected from the group consisting of: a monoclonal antibody against tissue cellular protein to be detected, polyclonal antibody against tissue cellular protein to be detected, and combinations thereof;
the colloidal silver antibody complex is formed by conjugating colloidal silver with an antibody selected from the group consisting of: a monoclonal antibody against tissue cellular protein to be detected, polyclonal antibodies against tissue cellular protein to be detected, and combinations thereof.

7. The test strip according to claim 1, wherein the testing points of the protein testing area are loaded with one or more antibodies selected from the group consisting of a monoclonal antibody against tissue proteins to be detected, polyclonal antibodies against tissue proteins to be detected and a combination thereof.

8. The test strip of claim 1, wherein the center distance of two adjacent testing points in each line is same.

9. The test strip of claim 1, wherein the vertical distance between adjacent lines is 1.0-1.8 mm, preferably 1.2-1.6 mm, and more preferably 1.3-1.5 mm.

10. The test strip of claim 1, wherein the distance between the center of two adjacent test points in one line and one test point in another adjacent line corresponding to the gap therebetween them is equal or unequal.

11. The test strip according to claim 1, wherein the tissue cellular protein to be detected is HER2 protein.

12. A test kit for testing tissue cellular protein, comprising:
(i) a test strip for testing tissue cellular protein according to claim 1;
(ii) a specification.

13. A use of a test strip according to claim 1 for detecting tissue cellular protein.

14. A method for testing tissue cellular protein which comprises: detecting a tissue cellular protein in a sample to be detected by using a test strip for tissue cellular protein detection according to claim 1.
